(19) United States
(12) Patent Application Publication
(10) Pub. No.: US 2019/0234965 A1
(43) Pub. Date: Aug. 1, 2019

(54) METHOD FOR DETERMINING RISKS ASSOCIATED WITH CARDIOVASCULAR DISEASES

(71) Applicant: OY MEDIX BIOCHEMICA AB, Espoo (FI)

(72) Inventors: Pirkko PUSSINEN, Helsingin yliopisto (FI); Timo SORSA, Helsinki (FI); Veikko SALOMAA, Helsinki (FI); Juuso JUHILA, Helsinki (FI); Armi KORVUO, Hyvinkaa (FI); Sinikka TIISALA, Helsinki (FI)

(21) Appl. No.: 16/337,440

(22) PCT Filed: Sep. 27, 2017
(86) PCT No.: PCT/FI2017/050680

§ 371 Date: Mar. 28, 2019

(30) Foreign Application Priority Data
Sep. 29, 2016 (FI) 20165730

(51) Int. Cl. G01N 33/68; G01N 33/573; G16H 50/30

(52) U.S. Cl. CPC G01N 33/6893; G01N 33/573; G16H 50/30; G01N 2333/96494; G01N 2800/32; G01N 2333/4737; G01N 2800/50

(57) ABSTRACT

The present invention relates to a novel method for determining risk of cardiovascular diseases comprising detecting of MMP-8 and CRP in a sample, and comparing the detected amounts with respective predetermined values of MMP-8 and CRP, wherein the detection of elevated levels of MMP-8 and CRP is indicative of presence or risk of cardiovascular event or disease. The present invention relates also to the use of detection of MMP-8 and CRP for predicting a risk for getting a cardiovascular event, for monitoring the effect of therapy on cardiovascular event or on cardiovascular disease, or for detecting the presence of a subclinical cardiovascular disease. Also, a method for constructing a risk prediction model for a presence of CVD disease or a risk of CVD events is presented.

### FIELD OF THE INVENTION

The present invention describes methods for improving prediction and estimating prognosis of cardiovascular diseases. The present methods are based on the identification and subsequent combination of biomarkers which are particularly well suited to discriminate between subjects in risk of cardiovascular disease events and healthy subjects. The biomarkers identified herein can also be used in detection of subclinical cardiovascular diseases and monitoring the effect of a treatment or medication on cardiovascular disease. The invention comprises the use of matrix metalloproteinase-8 (MMP-8) and C-reactive protein (CRP) for prediction and estimating prognosis of cardiovascular disease events, and also for monitoring the effects of treatments and medication on cardiovascular disease events. Further, MMP-8 and CRP concentration measurements can be used for detection of subclinical cardiovascular diseases.

### BACKGROUND

Cardiovascular diseases (CVDs) are a class of diseases involving the heart or blood vessels. Cardiovascular diseases are the leading cause of death globally. Cardiovascular diseases comprise such diseases as coronary artery disease (CAD), such as angina and acute myocardial infarction (AMI), stroke, hypertensive heart disease, rheumatic heart disease, cardiomyopathy, atrial fibrillation, congenital heart disease, endocarditis, aortic aneurysms, and peripheral artery disease.

Several distinct pathophysiological mechanisms play important roles in the CVD pathogenesis, development and course. These include, but are not limited to, inflammation, infections, prothrombotic and thrombotic activities, shear stress and endothelial responsiveness. Among the major causes of CVD is atherosclerosis, a disease characterized by an accumulation of lipids and inflammation in the affected vessel wall. During the course of atherosclerotic process and pathologic development, an affected arterial wall thickens due to accumulation and formation of fatty lesion or streak leading to build up of plaque (atheroma). Smooth muscle and foam cell core with extracellular lipid droplets and type I collagen rich ECM form a (fibrous) cap that by time and especially when affected by enhanced and continuous and sustained inflammation shall render to be prone to the rupture (Herman et al. 2001). These processes consequently lead to the occlusions and thrombi often responsible for the adverse cardiovascular event or outcomes.

Type I collagen is the major proteinous extracellular matrix (ECM) component and load bearing molecule of fibrous cap in the atherosclerotic lesions. Among the collagenolytic matrix metalloproteinases (MMPs) MMP-8 is catalytically the most efficient and competent to initiate the degradation of type I collagen (Sorsa et al. 2006).

Pathologically elevated MMP-8 mRNA and protein expression, production and serum/plasma levels have been found in unstable angina. In addition, associations between serum/plasma MMP-8 and course, as well as long-term development of adverse CVD outcomes, have been found. Elevated serum MMP-8 levels have been demonstrated to be related to and reflect an increased CVD morbidity. In cell culture studies, MMP-8 has been implicated in atherosclerotic plaque destabilization through its capacity to thin the protecting fibrous cap, thus rendering it more vulnerable to rupture (Herman et al. 2001). In human atherosclerotic plaque samples, MMP-8 protein and mRNA co-localize with macrophages (Molloy et al. 2004). In addition, abdominal aortic aneurysm contains significantly higher MMP-8 concentrations than normal aortic tissue (Wilson et al. 2005). Increased plaque MMP-8 activity has been observed in asymptomatic patients with plaque progression (Turu et al. 2005). Also plaques prone to rupture express more immunoreactive MMP-8 compared with lesions with more stable morphology (Herman et al. 2001).

Hitherto, however, only a few studies have investigated the associations of serum MMP-8 concentrations with CVDs. Results from two case-control studies with a small number of participants suggest that serum MMP-8 concentrations of patients with heart failure and cerebral ischemia are decreased (Wilson et al. 2005; Lorenz) et al. 2003). In the two most recent larger studies, the plasma MMP-8 concentration has been positively associated with the presence and severity of CAD (Kato et al. 2005) and with carotid artery plaque progression (Turu et al. 2005). The results of Tuomainen et al. (2007 and 2014) show that serum MMP-8 concentrations are elevated in prevalent or subclinical atherosclerosis and associate with fatal outcome. Plasma MMP-8 was recently found to be a significant predictor of metabolic syndrome and this relationship persisted even after adjusting for pro-inflammatory cytokines hs-CRP and TNF-α (Hoseini et al. 2015).

Elevated systemic MMP-8 also exerts significant roles in other diseases. The predominant role of MMP-8 in ECM processing and inflammatory and immune response modifications as well as being a drug target have been well documented.

CRP is a common inflammatory marker that has been found to be present in increased levels in patients who are at risk for cardiovascular disease. Recent research suggests that patients with elevated basal levels of CRP are at an increased risk of diabetes, hypertension and CVD. CRP is believed to be both a marker of atherosclerosis and coronary heart disease (CHD).

With this medical and biologic background the ability to identify and (PoC)-diagnose the early or initial onset and/or stages/steps/processes of persons at elevated risk of developing or progressing to adverse CVD outcome(s) is crucial and very important not only to the medical field and medical industry but also globally for the health care systems.

### BRIEF DESCRIPTION OF THE INVENTION

An objective of the invention was to provide a novel method for determining risk of cardiovascular disease comprising detecting Matrix Metalloproteinase-8 (MMP-8) and C-reactive protein (CRP) from a blood sample, and comparing the amounts of MMP-8 and CRP detected with respective predetermined values of MMP-8 and CRP, wherein the detection of elevated levels of MMP-8 and CRP is indicative of the presence of cardiovascular disease or indicative of the risk of cardiovascular event or cardiovascular disease.

Another objective of the present invention is a method for constructing a risk prediction model for presence of subclinical CVD disease before evident clinical symptoms or risk of CVD events, wherein said method is based on detection of MMP-8 and CRP in a sample.

Still another objective of the present invention was use of detecting MMP-8 and CRP for predicting a risk for getting a cardiovascular event, preferably within one year from the test; for evaluating the risk of a first or subsequent cardiovascular event; for monitoring the effect of therapy on cardiovascular event or on cardiovascular disease; or for detecting the presence of a subclinical cardiovascular disease before evident clinical symptoms.

According to one aspect of the invention is that based on detection of elevated MMP-8 and CRP levels a subject can be shown to additional tests or can be instructed to get further medical consultation.

According to a further aspect of the invention it will help to guide patient to cardiological examination before first or subsequent cardiovascular event.

### DESCRIPTION OF DRAWINGS

FIG. 1. Cumulative survival without incident CVD events (A) and AMI (B) in the follow-up of 1 year in subjects with (solid line) and without combination (dotted line) of high serum CRP and high MMP-8 concentrations. The analysis was done with Kaplan-Meier estimation adjusted for age and sex.

FIG. 2. Correlation data for MMP-8 concentrations obtained from patients with AMI and measured with time-resolved immunofluorometric assay (IFMA) and solid-phase enzyme-linked immunosorbent assay (ELISA). The results are presented in a scatter plot.

FIG. 3. Mean MMP-8 concentrations from patients with AMI or angina pectoris and from control subjects were measured with IFMA (A) and ELISA (B). The difference of MMP-8 concentrations between patients and controls is highly significant (p<0.001) with both assays but the difference between patients and controls is larger with IFMA than with ELISA. The results are presented as a box plot. The central line represents the mean, and the error bars represent the 95% Cl.

FIG. 4. Correlation data of MMP-8 concentrations from patients with angina pectoris or AMI and control subjects measured with IFMA (A) and ELISA (B) with CRP concentration. (A) MMP-8 concentration obtained with IFMA correlated statistically significantly with the correlation coefficient r 0.311 (p=0.008) while (B) the MMP-8 concentration obtained with ELISA correlated statistically significantly with the correlation coefficient r 0.301 (p=0.011).

### DETAILED DESCRIPTION OF THE INVENTION

The present inventors have found that the combination of two commonly known inflammatory markers that are presenting different immunological cascades in the human body, namely MMP-8 and CRP, can be used for prediction of the risk of cardiovascular diseases, estimating prognosis of cardiovascular diseases and monitoring the effectiveness of ongoing treatments and medication on cardiovascular diseases and on the risk of cardiovascular events. Further, MMP-8 and CRP can be used for detecting subclinical cardiovascular disease before evident clinical symptoms, such as angina, shortness of breath, fatigue, palpitations and light-headedness.

As such, the detection of MMP-8 and CRP concentrations in the whole blood, plasma or serum of a subject is useful for, e.g. 1) determining a risk of cardiovascular disease event; 2) determining the presence of subclinical cardiovascular disease or disorder before evident clinical symptoms; 3) estimating prognosis of a cardiovascular disease or disorder; and 4) monitoring the effectiveness of a treatment or medication on the progression of a cardiovascular disease or on the risk of having a cardiovascular event.

The combination of determining both CRP and MMP-8 seems to be useful. In CVD the atherosclerotic rupture processes, endothelial dysfunction and development of insulin receptor dysfunction involve the independent or co-operative action of pathologically excessive CRP, proinflammatory cytokines, reactive oxygen species and proteolysis. These mechanisms induce a continuous and sustained systemic low-grade inflammation, also called “a silent killer”. Proteolytic processes being part of the low-grade systemic inflammation involve the action of MMP-8, which in addition to being the most efficient type I collagenase can also degrade non-matrix bioactive substrates such as cytokines, chemokines, transforming growth factor-1, serpins, apolipoprotein A-I, insulin receptor, immune and cell signaling factors thereby modifying a systemic immune and metabolic responses to pathologic courses/directions in the various diseases.

MMP-8 can be expressed and produced by various cells including—but not limited to—neutrophils, monocyte/macrophages, endothelial cells, fibroblasts, epithelial cells and plasma cells. Many of these cells are present in or are recruited to the atherosclerotic or CVD lesions. These cells affect CRP and proinflammatory mediators to be expressed and also produce pathologically elevated systemic MMP-8 that is often detected as well as regarded to be an essential player of the systemic low grade inflammation.

Cardiovascular diseases according to the invention comprise such diseases as coronary artery disease (CAD), such as angina pectoris and acute myocardial infarction (AMI), stroke, hypertensive heart disease, rheumatic heart disease, cardiomyopathy, atrial fibrillation, congenital heart disease, endocarditis, aortic aneurysms, and peripheral artery disease. In preferred embodiments of the invention the disease is a CVD or a disease event, for example CAD event, such as AMI.

An embodiment of the invention relates to a method for determining risks associated with cardiovascular diseases comprising detecting MMP-8 and CRP in a sample, and comparing the amounts of MMP-8 and CRP with respective predetermined values of MMP-8 and CRP, wherein the detection of elevated levels of MMP-8 and CRP are indicative of the presence of cardiovascular disease or indicative of a risk of cardiovascular event or cardiovascular disease in a subject. Based on detection of elevated MMP-8 and CRP levels the subject can be instructed to seek further medical consultation or additional examinations.

A further preferred embodiment of the invention relates to a method for detecting cardiovascular diseases, evaluating the risk of a first or subsequent cardiovascular event, detecting subclinical cardiovascular diseases before evident clinical symptoms, or monitoring the effectiveness of a treatment or medication on the progression of cardiovascular disease or on the risk of having a cardiovascular event, said method comprising detecting MMP-8 and CRP in a sample, and comparing the amounts of MMP-8 and CRP detected with respective predetermined values of MMP-8 and CRP, wherein the detection of elevated levels of MMP-8 and CRP is indicative of the presence of cardiovascular disease or indicative of the risk of cardiovascular event or cardiovascular disease. According to the present invention, the detected levels of MMP-8 and CRP are elevated when the amount of MMP-8 is above the predetermined value for MMP-8 and the amount of CRP is above the predetermined value for CRP.

According to the present invention in a method for determining risks associated with cardiovascular diseases, the cardiovascular event or cardiovascular disease can be selected from the list consisting of cardiovascular disease (CVD), coronary artery disease (CAD), such as angina pectoris and acute myocardial infarction (AMI), stroke, hypertensive heart disease, rheumatic heart disease, cardiomyopathy, atrial fibrillation, congenital heart disease, endocarditis, aortic aneurysms, and peripheral artery disease, preferably said cardiovascular event or cardiovascular disease is a CVD event or a CAD, such as AMI.

Risk prediction models can be used to estimate the probability of either having (diagnostic model) or developing a particular disease or outcome (prognostic model). In clinical practice, these models are used to inform patients and guide therapeutic management. According to Hendriksen et al. (2013) three phases are recommended before a prediction model may be used in daily practice. In the development phase, the focus is on model development commonly using a multivariable logistic (diagnostic) or survival (prognostic) regression analysis. The performance of the developed model is expressed by discrimination, calibration and (re-)classification. In the validation phase, the developed model is tested in a new set of patients using these same performance measures. Finally, in the impact phase the ability of a prediction model to actually guide patient management is evaluated. MMP-8 and CRP values detected with the method as described herein can be used for constructing prediction models for risk of CVD events.

Treatments for cardiovascular disease may include lifestyle changes, medications, invasive procedures, such as revascularizations cardiac rehabilitation, or combinations thereof.

Medicines for treating cardiovascular diseases include: antiplatelets that thin blood and prevent it clotting, statins such as atorvastatin, simvastatin, rosuvastatin and pravastatin that lower cholesterol, beta-blockers—including atenolol, bisoprolol, metoprolol and nebivolol, nitrates, ACE (angiotensin-converting enzyme) inhibitors, such as ramipril and lisinopril, angiotensin II receptor antagonists and calcium channel blockers , diuretics that work by flushing excess water and salt from the body through urine, as well as doxycycline medication that reduces elevated CRP and MMP-8 and MMP-9 levels in plasma or serum (Payne et al. 2011, Kormi et al. 2014, Alfakry et al. 2016). The method of the present invention can be used also to monitor the effectiveness of these or other treatments on a cardiovascular disease and for predicting the first or subsequent cardiovascular event during the treatment. Based on the results, the disease of the patient is under control and the patient is at low risk, when the patient, due to treatment and medication procedures, has low MMP-8 and CRP values and also the combination of MMP-8 and CRP values is low due to treatment and medication procedures.

The sample used for detecting or determining the MMP-8 and/or CRP concentration, amount or level is typically whole blood, plasma or serum. In certain instances, the method of the present invention further comprises obtaining the sample from the individual prior to detecting or determining the presence, amount or level of the marker in the sample. Preferably, the sample is serum or plasma.

MMP-8 concentration in the sample can be measured using any method known in the art. The assay can be qualitative, semi-quantitative or quantitative immunoassay. Non-limiting examples of suitable detection methods according to the invention include Western blotting, IFMA, EIA, ELISA, IEMA, Lateral Flow Assay, Dip-stick assay, microfluidics point-of-care (PoC) assay, surface plasmonic resonance assay, electrochemical assay or any other known ligand binding or direct detection assay system. The direct detection assay systems or technologies mean any method that is not based on ligand binding for analysis, i.e., technologies like; Size Exclusion Chromatography [SEC], such as High Pressure Liquid chromatography [HPLC] or Gel Permeation chromatography (GPC) such as SDS-PAGE; or molecular spectroscopy methods, such as Nuclear Magnetic Resonance Spectroscopy (NMR), UV/VIS-Spectroscopy, Electrospray-Ionisation (ESI) etc.

According to one aspect of the present invention the detection of MMP-8 and CRP can be performed with immunoassay. More preferably, one or more immunoassays can be selected from the group consisting of ELISA, IFMA, turbidimetry, nephelometry, particle enhanced turbidimetry, particle enhanced nephelometry, latex agglutination, lateral flow assay and microfluidics PoC assay.

A preferred embodiment of the present invention is the method for predicting a cardiovascular event or estimating prognosis of a cardiovascular disease, monitoring the effectiveness of a treatment or medication on the progression of cardiovascular disease and on the risk of having a cardiovascular event and detection of subclinical cardiovascular diseases before evident clinical symptoms, wherein CRP is tested for example by Latex immunoassay CRP16 applying a cut-off-value at approximately 2.5 mg/I and MMP-8 is tested by a time-resolved immunofluorometric assay applying a cut-off-value at approximately 55 ng/ml.

Unless otherwise specified, the terms, which are used in the specification and claims, have the meanings commonly used in the field of diagnostics. Specifically, the following terms have the meanings indicated below.

The term detecting subclinical disease or detecting subclinical disorder should be understood to mean identification or determining of the presence of a subclinical disease, before evident clinical symptoms, i.e. diagnosis of the disease or disorder. The term subclinical disease should be understood to mean an illness that is staying below the surface of clinical detection. A subclinical disease has no recognizable clinical findings. It is distinct from a clinical disease, which has signs and symptoms that can be recognized. Many diseases, including CVD, diabetes, hypothyroidism, and rheumatoid arthritis, are frequently subclinical before they surface as clinical diseases.

The terms positive and negative refer to values of a test analyte, i.e. MMP-8 or CRP, concentrations in a sample to be above (high or positive) and below (low or negative) a predetermined value (baseline, threshold or reference concentration), respectively. The predetermined value for an analyte in a sample refers to the base or threshold concentration of an analyte in a sample in normal individuals; and if the value of the analyte in said sample is above such predetermined value, the test result is positive. The predetermined value for an analyte in a sample may vary depending on the format of the assay, and the specific reagents employed in the assay (e.g., the particular antibodies used), but can be determined and set by those skilled in the art by assessing the concentration of the analyte in a sample in normal individuals relative to control samples containing known amounts of the analyte.

A continuous variable refers to a variable that can take any value between its minimum value and its maximum value.

Active MMP-8 refers to the different forms of activated proteinase differing from its pro- or precursor forms.

MMP-8 activation refers to biological or biochemical processes of transforming and/or converting preforms of MMP-8 to active/activated i.e. catalytically competent MMP-8. According to one preferred embodiment of the invention activated MMP-8 is detected.

The present inventors have earlier found (WO 2015/128549) that by detecting smaller MMP-8 fragments, instead of the high molecular weight species of active MMP-8, the detection of active MMP-8 can be enhanced.

Embodiments of the invention also provide for systems and computer readable medium for causing computer systems to perform a method for determining whether an individual has a risk associated with evolving a cardiovascular disease or event, based on determining MMP-8 and CRP.

Especially the invention further relates to a system for analyzing a biological sample comprising:
a) a determination module configured to receive a biological sample and to determine
a MMP-8 and CRP; and/or
b) a test result information, wherein the test result information comprises MMP-8 and CRP values
c) a storage device configured to store information from the determination module;
d) a comparison module adapted to compare the test result information stored on the storage device with reference data, and to provide a comparison result, wherein the comparison result is derived from a reference sample/predetermined level which is derived from;
a subject or a patient group known to currently have a normal level of MMP-8 whereby similar results for the biological sample and the reference sample are indicative for the subject currently to not have or not be predisposed to the disease or to a disease event or not have or not be predisposed to a risk of developing a disease or disease event or progressing the disease; and/or
a subject or a patient group known to have the disease or be predisposed to the disease whereby similar results for the biological sample and the reference sample are indicative for the subject to have or be predisposed to the disease or to the disease event or to have or to be predisposed to a risk of developing a disease or disease event or progressing the disease, and
e) a display module for displaying a content based in part on the comparison result for the user, wherein the content is a signal indicative for the subject to currently have a disease or to be predisposed to a cardiovascular disease or to be predisposed to have an increased risk of developing a disease or disease event or progressing a disease.

### EXAMPLES

The following examples are given solely for the purpose of illustrating various embodiments of the invention and they are not meant to limit the present invention in any way. One skilled in the art will appreciate readily that the present invention which is defined by the accompanied claims is well adapted to carry out the objects and obtain the ends and advantages mentioned above.

Population-Based Sample

The FINRISK97 involved a population-based sample of 8446 25-74 year old participants of the survey, which was conducted in five geographical areas in Finland (Borodulin et al. 2015). The survey included a self-administered questionnaire and a clinical examination with weight, height, and blood pressure measurements as well as blood drawing. The study was approved by the Ethics Committee of the National Public Health Institute and conducted according to the Helsinki Declaration.

Laboratory Analyses

Before blood sampling, the participants were asked to fast for 4 hours and to avoid heavy meals earlier during the day. The median fasting time was 5 (IQR 2) hours. Measurement of ultrasensitive CRP was carried out from frozen serum samples (−70° C.) using a latex immunoassay (Sentinel diagnostics, Milan, Italy) on Architect c8000 analyzer (Abbott Laboratories, Abbott Park, Ill., USA) at the Disease Risk Unit in the National Institute for Health and Welfare, Helsinki in 2005. The concentration of MMP-8 was determined by IFMA (Medix Biochemica, Espoo, Finland) according to manufacturer's instructions.

MMP-8 Analysis with IFMA

MMP-8 IFMA is a quantitative enzyme immunoassay for the determination of human MMP-8. This sandwich assay uses two monoclonal antibodies against human MMP-8. Antibodies 1491-E6-F7 and 1492-B3-C11 (Medix Biochemica, Espoo, Finland) were used as a catching antibody and a tracer antibody, respectively. Microwell plates are coated with one monoclonal antibody against MMP-8. The other antibody is conjugated to HRP forming the enzyme conjugate used to detect the presence of MMP-8. To run the assay, 80 μl of Assay Buffer and 20 μl of standards, controls and samples are added to appropriate wells of the plate. The plate is incubated for one hour at room temperature on a horizontal shaker. MMP-8 in standards, controls, and if present in samples, is bound to the microwells. The wells are washed five times in order to remove unbound substances. After this washing step, 100 μl of the enzyme conjugate is added to all wells. The plate is incubated again for one hour on a horizontal shaker and washed as above. Thereafter, 100 μl of ABTS enzyme substrate is added to the wells. The plate is shaken as above for 15 minutes. The reaction is terminated by adding 50 μl of an acidic stopping solution. To mix the solutions, the plate is gently shaken. The absorbance of the solutions in the wells is measured at 414 nm using a microplate reader (Multiskan, Thermo Fisher Scientific, Vantaa, Finland). The concentrations of controls and samples are obtained from the standard curve created.

MMP-8 Analysis with ELISA (Amersham)

ELISA is a ready-to use solid-phase enzyme-linked immunosorbent assay based on the sandwich principle. 100 μl samples (dilution 1:4) and standards are incubated one hour in room temperature in microtiter wells coated with antibodies recognizing human MMP-8. After incubation the wells are washed four times. 100 μl biotinylated tracer antibody is added that will bind to the captured human MMP-8. After one hour incubation the wells are washed four times. Then 100 μl streptavidin-peroxidase conjugate is added to bind to the biotinylated tracer antibody. After one hour incubation the wells are washed again. 100 μl TMB solution is added, streptavidin-peroxidase conjugate will react with that substrate, tetramethylbenzidine (TMB). The 30 min incubation is stopped by the 100 μl addition of oxalic acid. The absorbance at 450 nm is measured with a spectrophotometer (Multiskan, Thermo Fisher Scientific, Vantaa, Finland). The human MMP-8 concentration of samples, which are run concurrently with the standards, can be determined from the standard curve.

hsCRP Analysis

hsCRP analysis was done using Latex immunoassay CRP16 (Abbott, Architect c8000) as described in Salomaa et al. 2010.

Statistics

The following endpoints within one year were ascertained through the record linkage of the National Causes of Death Register and the National Hospital Discharge register: cardiovascular disease (CVD), acute myocardial infarction (AMI), inflammatory bowel disease (IBD) (follow-up for 5 years due to low incidence), and cancer (except non-melanoma skin cancer). The analyses were done on 7448, 7893, or 8276 subjects who were free from CVD, IBD, or cancer, respectively, at baseline.

The statistical significance of the differences in the serum CRP and MMP-8 concentrations between the subjects with and without incident disease or event was analyzed with the t-test. Before the analyses, values with skewed distribution were normalized by logarithmic transformation. The survival data for incident diseases taking into account the MMP-8 and CRP concentrations was analyzed by using the Cox proportional hazards model adjusted for age and gender. The hazards were estimated for the percentiles of MMP-8 and CRP concentrations and the 50th percentile was chosen as the cut-off value, i.e. the reference category was persons with either MMP-8 or CRP value or both values below the 50^{th} percentile. The results were thus calculated for subjects, whose MMP-8 and CRP concentrations both exceeded the threshold compared to the reference category. The statistical analyses were performed using SPSS 22.0 (IBM Corp. Released 2013. IBM SPSS Statistics for Windows, Version 22.0. Armonk, NY: IBM Corp.

### Example 1

### Serum MMP-8 and CRP Concentrations Associated with CVD and AMI in Subjects

Measuring of concentration levels of serum MMP-8 and CRP concentrations in subjects free from CVD at baseline was performed and the associations of these concentrations with the possibility to have a CVD or AMI were determined with 1 year follow-up, as described above. The results showed that the sum of serum CRP and MMP-8 concentrations—both being higher than the respective threshold/the mean value—was higher in persons experiencing an AMI or any CVD event compared to those who did not. This difference in combination between these groups was significant (Tables 1 and 2). As shown in Table 1, the mean concentration sum for subjects with CVD was 1.97, while in subjects without CVD it was 1.55 (p<0.001). The mean concentration of MMP-8 was not significant alone, while CRP concentration was significant also when considered alone for CVD (p <0.001). In Table 2 it is shown that the mean concentration sum for subjects with AMI was 2.07, while in subjects without AMI it was 1.55 (p=0.001). The mean concentration of neither MMP-8 nor CRP was significant alone in AMI.

TABLE 1
Mean serum MMP-8 and CRP concentrations in subjects
free from CVD at baseline but with and without an
incident CVD event in the follow-up of 1 year.

| | Without CVD event | With CVD event | | |
|---|---|---|---|---|
| | Mean (SD) | | | p-value ¹ |
| CRP (mg/l) | 2.40 (4.88) | 10.8 | (21.1) | <0.001 |
| MMP-8 (ng/ml) | 50.3 (66.7) | 66.5 | (105.9) | 0.456 |
| Log CRP + log | 1.55 (0.67) | 1.97 | (1.02) | <0.001 |
| MMP-8 | | | | |

¹ t-test after logarithmic transformation.

TABLE 2
Mean serum MMP-8 and CRP concentrations in subjects
free from CVD at baseline but with and without
an AMI event in the follow-up of 1 year.

| | Without AMI | With AMI | | |
|---|---|---|---|---|
| | Mean (SD) | | | p-value ¹ |
| CRP (mg/l) | 2.43 (4.98) | 17.0 | (21.1) | 0.051 |
| MMP-8 (ng/ml) | 50.3 (66.7) | 84.5 | (142.4) | 0.272 |
| Log CRP + log MMP-8 | 1.55 (0.68) | 2.07 | (1.23) | 0.001 |

¹ t-test after logarithmic transformation.

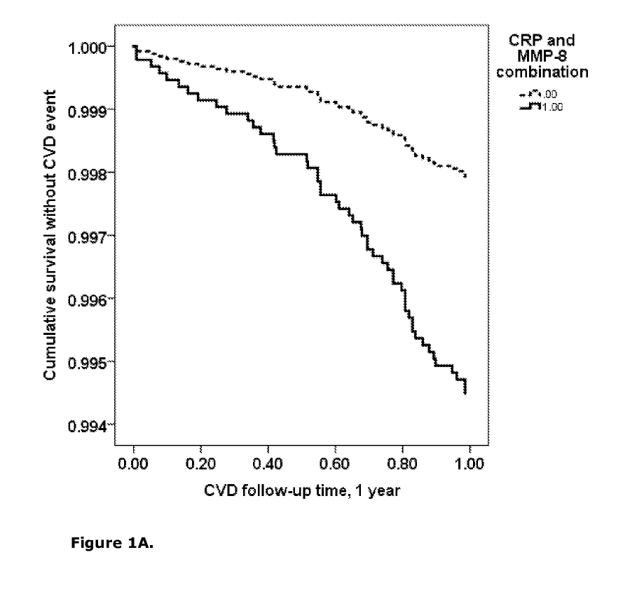

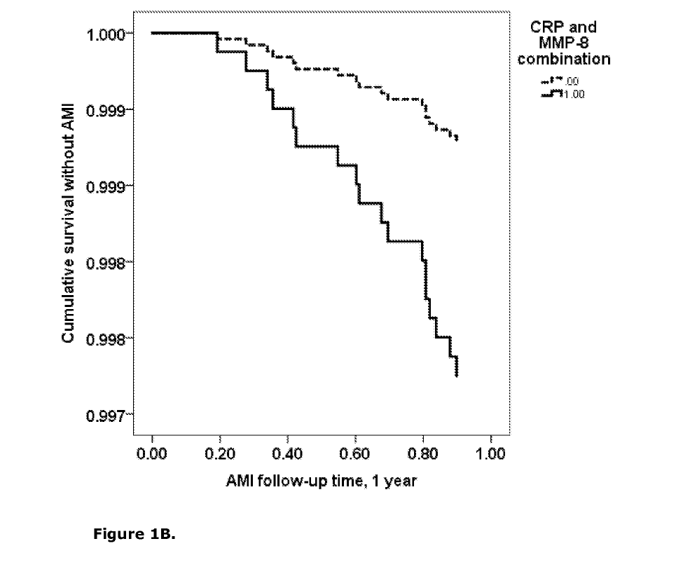

Table 3 discloses hazard ratios (HRs) for incident CVD events as calculated from high (above mean or positive) MMP-8 and CRP concentration values compared to low (below mean or negative) values, wherein HR of the reference group (below mean or negative) was set to 1. With all MMP-8, CRP or a combination thereof values above mean the HR appeared to be higher than 1. The HR was higher with combination of CRP and MMP-8 (values) than with either alone. Combination of high (above the 50^{th} percentile) CRP and high MMP-8 concentrations tended to show higher HRs than a high concentration of either of these biomarkers alone. The combination results showed a statistical significance in risk prediction, p values being 0.011 for CVD and 0.043 for AMI, respectively. In FIGS. 1A and 1B the cumulative survival without a CVD event or an AMI are presented for those with both CRP and MMP-8 above the 50^{th} percentile (marked 1.0) compared to those with either MMP-8 or CRP or both MMP-8 and CRP being below the 50^{th} percentile (marked 0). The figures indicate a higher risk for those subjects with both CRP and MMP-8 above the 50^{th} percentile.

TABLE 3
Association of high serum CRP and MMP-8 concentrations
and their combination with incident CVD events and
AMI in the follow-up of 1 year. Mean values are
2.50 mg/l for CRP and 55.0 ng/ml for MMP-8.

| | HR (95% Cl) | | |
|---|---|---|---|
| | Below mean | Above mean | p-value ¹ |
| CVD event | | | |
| CRP | 1 | 2.03 (1.17-3.51) | 0.011 |
| MMP-8 | 1 | 1.45 (0.56-3.75) | 0.439 |
| Combination CRP, MMP-8 | 1 | 2.67 (1.34-5.34) | 0.005 |
| AMI | | | |
| CRP | 1 | 1.50 (0.58-3.90) | 0.401 |
| MMP-8 | 1 | 1.59 (0.59-4.48) | 0.380 |
| Combination CRP, MMP-8 | 1 | 3.15 (1.04-9.57) | 0.043 |

¹ Cox regressions adjusted for age and sex, p-values for estimates for concentrations above mean.

### Example 2

### Serum MMP-8 and CRP Concentrations Associated with Inflammatory Bowel Disease (IBD) and Cancer

Inflammatory bowel disease (IBD) is a group of inflammatory conditions of the colon and small intestine. Crohn's disease and ulcerative colitis are the principal types of inflammatory bowel disease. IBD was earlier shown to associate significantly with elevated CRP due to the various roles this protein can assume in affected patients (Henriksen et al. 2008). As an inflammatory marker, CRP helps to predict, monitor, and evaluate IBD in terms of its presence, severity, and therapeutics.

For example Danish researchers have shown that people with high blood levels of CRP have a 30 percent greater risk of developing any cancer later in life, and were associated with the risk of developing lung and possibly colorectal cancers, compared with people with low CRP levels (Cancer.Net, ASCO's Patient Web site). Researchers have also found that among people with cancer, those with high CRP levels prior to their diagnosis were 80 percent more likely to die sooner than people with cancer who did not have elevated CRP.

The previous studies thus suggest a connection between CRP and IBD and CRP and cancer. The present inventors wanted to study whether MMP-8 concentration and/or the sum of MMP-8 and CRP concentrations could be used as indicative or predictive markers regarding these diseases. In the present studies (Table 4) the mean CRP or MMP-8 concentrations did not significantly differ between subjects with and without incident IBD, but the sum of these markers did. The sum was also higher in subjects getting incident cancer than those who did not, and this difference was due to higher CRP levels. For cancer, the mean CRP concentration was significant alone.

TABLE 4
Serum MMP-8 and CRP concentrations and their sums in subjects
with and without IBD or cancer in the follow-up of 5 year.

| | Mean (SD) | | |
|---|---|---|---|
| | Without IBD | With IBD | p-value ¹ |
| CRP (mg/l) | 2.54 (5.28) | 3.68 (3.34) | 0.083 |
| MMP-8 (ng/ml) | 50.0 (66.5) | 68.4 (53.4) | 0.057 |
| Log CRP + log MMP-8 | 1.57 (0.68) | 2.04 (0.57) | 0.017 |
| | Without cancer | With cancer | |
| CRP (mg/l) | 2.48 (5.09) | 3.50 (6.52) | <0.001 |
| MMP-8 (ng/ml) | 49.8 (66.1) | 54.3 (72.9) | 0.709 |
| Log CRP + log MMP-8 | 1.56 (0.68) | 1.74 (0.73) | <0.001 |

¹ t-test after logarithmic transformation.

The association of these biomarkers with incident cancer in a follow-up of one year was also examined. In this case, CRP and MMP-8 concentrations appeared to be significantly higher both separately and in combination in subjects with incident cancer than in subjects without cancer (Table 5).

TABLE 5
Serum MMP-8 and CRP concentrations and their sum in subjects
with and without cancer in the follow-up of 1 vear.

| | Mean (SD) | | |
|---|---|---|---|
| | Without cancer | With cancer | p-value ¹ |
| CRP (mg/l) | 2.49 (5.10) | 4.97 (9.55) | 0.024 |
| MMP-8 (ng/ml) | 49.8 (66.1) | 66.1 (77.1) | 0.048 |
| Log CRP + log MMP-8 | 1.56 (0.68) | 1.89 (0.83) | 0.017 |

¹ t-test after logarithmic transformation

High MMP-8 concentration alone was significantly associated with the risk of incident cancer. However, combining high (above 50^{th} percentile) CRP and high (above 50^{th} percentile) MMP-8 concentrations did not improve prediction of incident IBD or cancer (Table 6) over the prediction obtained with MMP-8 alone. Neither of the markers nor their sum was associated with the risk of IBD.

TABLE 6
Association of high serum CRP and MMP-8 concentrations and
their combination with incident IBD in the follow-up of 5
years and incident cancer in the follow-up of 1 year.

| | HR (95% Cl) | | |
|---|---|---|---|
| | Below mean | Above mean | p-value ¹ |
| IBD | | | |
| CRP | 1 | 2.15 (0.67-6.91) | 0.199 |
| MMP-8 | 1 | 2.57 (0.86-7.68) | 0.092 |
| Combination CRP, MMP-8 | 1 | 2.32 (0.51-10.6) | 0.278 |
| Cancer | | | |
| CRP | 1 | 1.36 (0.73-2.52) | 0.337 |
| MMP-8 | 1 | 2.46 (1.36-4.43) | 0.003 |
| Combination CRP, MMP-8 | 1 | 2.41 (1.12-5.18) | 0.025 |

¹ Cox regressions adjusted for age and sex, p-values for estimates for concentrations above mean.

### Example 3

### Correlation Data about MMP-8 Concentration Determined by IFMA or ELISA

Knowing the variation between different antibodies and methods known in the field used for detecting MMP-8 and activated parts of MMP-8, the inventors wanted to study whether the correlation data obtained was dependent on an assay used for determining MMP-8 concentration. Measuring MMP-8 concentration was done by IFMA and ELISA as described earlier using different MMP-8 antibodies. The measurements were done for patients (343 patients, who were admitted for Acute Coronary Syndrome (ACS) and control subjects (Pussinen et al. 2013). Control subjects were matched with age ±2 years, sex, and parish. Inclusion criteria were: no history of definite or suspected CHD or stroke, and no operations or chemotherapy within the previous 4 weeks. They did not have a positive history of angina i.e. chest pain in any location related to exercise and relieved by rest. None of them had any medication for diabetes, hypertension, or dyslipidemia.

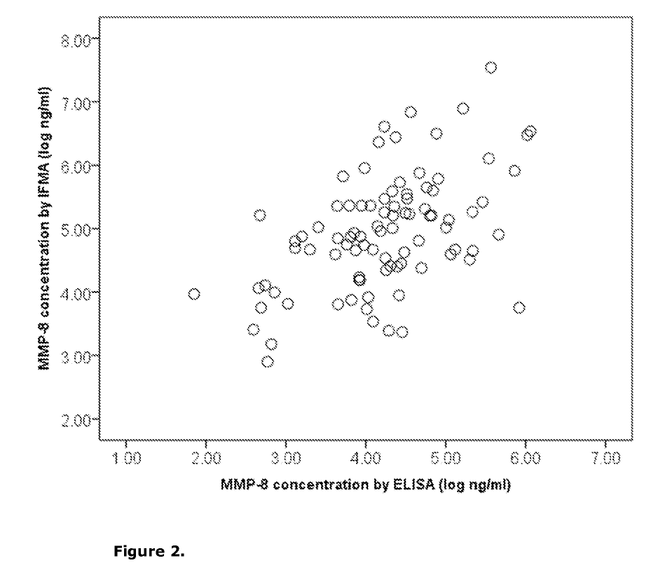

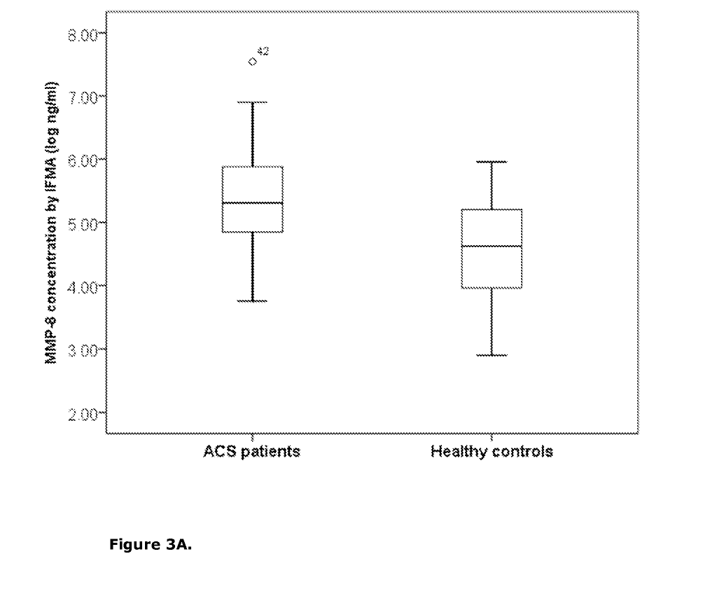

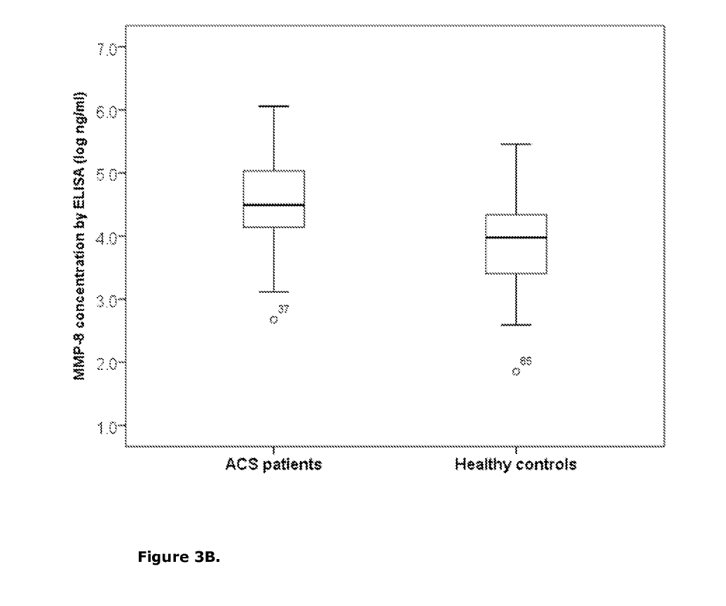

The results are presented both in Table 7 and scatter plot (FIG. 2). When mean MMP-8 concentration levels were measured with IFMA and ELISA, the difference of MMP-8 levels between patients and control subjects with both assays was highly significant (p<0.001). The difference between patients with angina pectoris or AMI and control subjects was bigger with IFMA than with ELISA (FIG. 3 and Table 8).

TABLE 7
Correlation data for MMP-8 concentrations obtained
from patients with AMI and measured with IFMA and ELISA.
Pearson correlation for logarithmically transformed
concentrations, r = correlation coefficient.

| | MMP-8-IFMA |
|---|---|
| MMP-8-ELISA | r = 0.509 |
| | p < 0.001 |
| | n = 90 |

TABLE 8
Mean MMP-8 concentrations measured with IFMA and ELISA.

| Assay | Group | N | Mean | SD | SE |
|---|---|---|---|---|---|
| MMP-8, IFMA (ng/ml) | ACS-patients | 45 | 315.6 | 337.2 | 50.3 |
| | Controls | 45 | 117.5 | 85.0 | 12.7 |
| MMP-8, ELISA (ng/ml) | ACS-patients | 45 | 126.6 | 104.0 | 15.5 |
| Amersham | Controls | 45 | 65.6 | 51.8 | 7.7 |

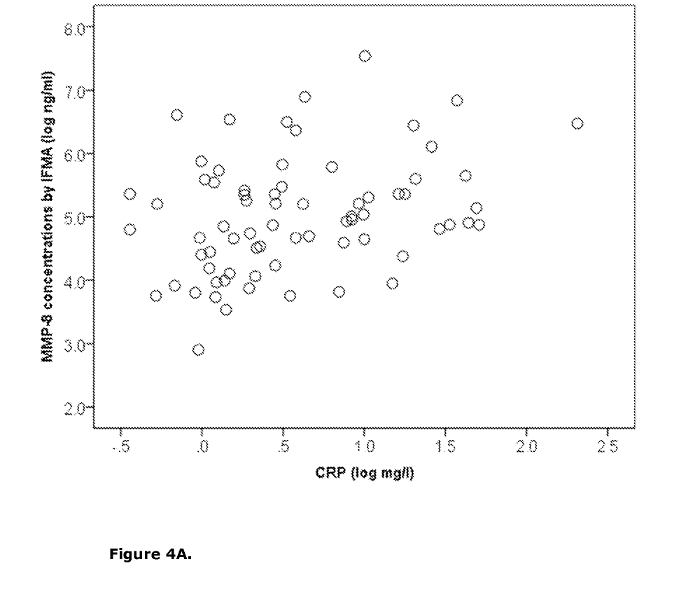

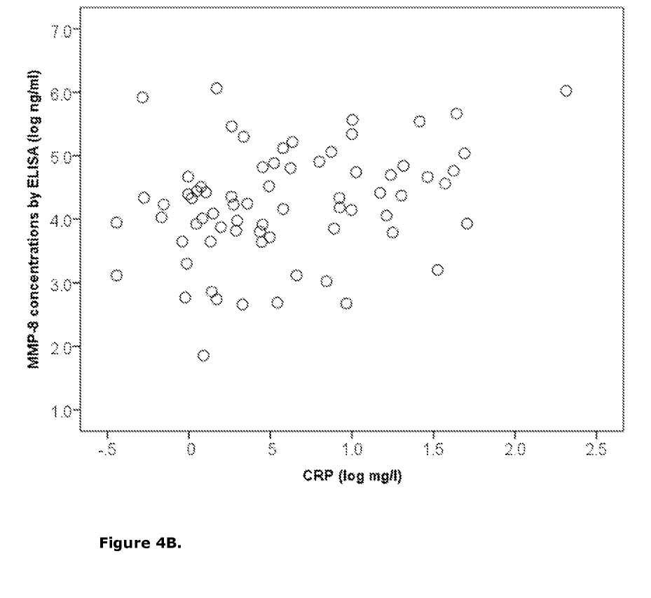

MMP-8 concentration was measured by IFMA (FIG. 4A) and ELISA (FIG. 4B) from patients with angina pectoris or AMI and control subjects and the obtained concentrations were correlated with CRP concentration. It was shown that (A) MMP-8 concentrations measured with IFMA correlated statistically significantly with the correlation coefficient r 0.311 (p=0.008), while (B) the MMP-8 concentrations measured with ELISA correlated statistically significantly with the correlation coefficient r 0.301 (p=0.011) to the CRP concentration (FIG. 4). The correlation appears thus to be test type independent. The mean MMP-8 concentrations (with standard deviations) measured with IFMA or ELISA are presented in Table 9.

TABLE 9
The mean MMP-8 concentrations (with SD) obtained with IFMA or
ELISA. The concentrations are logarithmically transformed.

| Assay | Group | Mean | SD |
|---|---|---|---|
| MMP-8, IFMA (ng/ml) | Controls | 4.57 | 0.70 |
| | Angina pectoris | 5.40 | 1.11 |
| | AMI | 5.44 | 0.75 |
| | Total | 5.04 | 0.89 |
| MMP-8, ELISA (ng/ml) | Controls | 3.86 | 0.81 |
| Amersham | Angina pectoris | 4.39 | 0.79 |
| | AMI | 4.65 | 0.81 |
| | Total | 4.26 | 0.88 |

### REFERENCES

Alfakry H, Malle E, Koyani C N, Pussinen P J, Sorsa T. Neutrophil proteolytic activation cascades: a possible mechanistic link between chronic periodontis and coronary heart disease. Innate Immunity 2016; 22(1):85-99.

Borodulin K, Vartiainen E, Peltonen M, Jousilahti P, Juolevi A, Laatikainen T, Männistö S, Salomaa V, Sundvall J, Puska P. Forty-year trends in cardiovascular risk factors in Finland. Eur J Public Health 2015; 25(3):539-46.

Dejonckheere E. et al., Matrix metalloproteinase-8 has a central role in inflammatory disorders and cancer progression. Cytokine & Growth Factor Reviews 2011; 22:73-81.

Hendriksen J M, Geersing G J, Moons K G, de Groot J A. Diagnostic and prognostic prediction models. J Thromb Haemost. 2013; Suppl 1:129-41.

Henriksen_M, Jahnsen J, Lygren I, Stray N, Sauar J, Vatn MH, Moum B; IBSEN Study Group. C-reactive protein: a predictive factor and marker of inflammation in inflammatory bowel disease. Results from a prospective population-based study. Gut 2008; 57(11):1518-23.

Herman M P, Sukhova G K, Libby P, Gerdes N, Tang N, Horton D B, et al. Expression of neutrophil collagenase (matrix metalloproteinase-8) in human atheroma: a novel collagenolytic pathway suggested by transcriptional profiling. Circulation 2001; 16(104):1899-904.

Hoseini S M, Kalantari A, Afarideh M, et al. Evaluation of plasma MMP-8, MMP-9 and TIMP-1 identifies candidate cardiometabolic risk marker in metabolic syndrome: results from double-blinded nested case-control study. Metabolism. 2015; 64:527-38.

Kato R, Momiyama Y, Ohmori R, Taniguchi H, Nakamura H, Ohsuzu F. Plasma matrix metalloproteinase-8 concentrations are associated with the presence and severity of coronary artery disease. Circ J. 2005; 69:1035-1040.

Kormi I, Alfakry H, Tervahartiala T, Pussinen PJ, Sinisalo J, Sorsa T. The effect of prolonged systemic doxycycline therapy on serum tissue degrading proteinases in coronary bypass patients: a randomized, double-masked, placebo-controlled clinical trial. Inflamm Res. 2014; 63:329-334.

Lorenzl S, De Pasquale G, Segal A Z, Beal M F. Dysregulation of the levels of matrix metalloproteinases and tissue inhibitors of matrix metalloproteinases in the early phase of cerebral ischemia. Stroke. 2003; 34:e37-e38.

Molloy K J, Thompson M M, Jones J L, Schwalbe E C, Bell P R F, Naylor A R, Loftus I M. Unstable carotid plaques exhibit raised matrix metalloproteinase-8 activity. Circulation. 2004; 110:337-343.

Payne J B, Golub L M, Stoner J A, Lee H, Reinhardt R A, Sorsa T, Slepian M L. The effect of subantimicrobial-dose-doxycycline periondotal therapy on serum biomarkers of systemic inflammation: a randomized, double-masked, placebo-controlled clinical trial. J Am Dent Assoc. 2011; 152(3):262-273.

Pussinen P J, Sarna S, Puolakkainen M, Ohlin H, Sorsa T, Pesonen E. The balance of serum matrix metalloproteinase-8 and its tissue inhibitor in acute coronary syndrome and its recurrence. Intl Cardiol. 2013; 167(2):362-8.

Salomaa V, Havulinna A, Saarela O, Zeller T, Jousilahti P, Jula A, Muenzel T, Aromaa A, Evans A, Kuulasmaa K, Blankenberg S. Thirty-one novel biomarkers as predictors for clinically incident diabetes. PLoS One. 2010; 5(4):e10100.

Sorsa T, Tjäderhane L, Konttinen Y T, Lauhio A, Salo T, Lee H M, et al. Matrix metalloproteinases: contribution to pathogenesis, diagnosis and treatment of periodontal inflammation. Ann Med. 2006; 38:306-21.

Tuomainen A M, Nyyssonen K, Laukkanen J A, Tervahartiala T, Tuomainen T P, Salonen J T, Sorsa T, Pussinen P J. Serum matrix metalloproteinase-8 concentrations are associated with cardiovascular outcome in men. Arterioscler Thromb Vasc Biol. 2007; 27:2722-2728.

Tuomainen A M, Kormi I, Havulinna A S, Tervahartiala T, Salomaa V, Sorsa T, Pussinen P J. Serum tissue-degrading proteinases and incident cardiovacular disease events. Eur J Prey Cardiol. 2014; 21(7):806-812.

Turu M M, Krupinski J, Catena E, Rosell A, Montaner J, Rubio F, Alvarez-Sabin J, Cairols M, Badimon L. Intraplaque MMP-8 levels are increased in asymptomatic patients with carotid plaque progression on ultrasound. Atherosclerosis. 2005; 187:161-169.

Wilson E M, Gunasinghe H R, Coker M L, Sprunger P, Lee-Jackson D, Bozkurt B, Deswal A, Mann D L, Spinale F G. Plasma matrix metalloproteinase and inhibitor profiles in patients with heart failure. J Card Fail. 2002; 8:390-398.

Wilson W R, Schwalbe E C, Jones J L, Bell P R, Thompson M M. Matrix metalloproteinase 8 (neutrophil collagenase) in the pathogenesis of abdominal aortic aneurysm. Br J Surg. 2005; 92:828-833.

1. A method for monitoring the effect of a treatment or medication on cardiovascular disease in a subject, the method comprising
administering a treatment or medication to the subject,
detecting Matrix Metalloproteinase-8 (MMP-8) and C-reactive protein (CRP) in a sample from the subject, and
comparing the amounts of MMP-8 and CRP detected with respective predetermined values of MMP-8 and CRP, wherein the detection of lowered levels of MMP-8 and CRP is indicative of the treatment or medication being effective.

2. The method according to claim 1, wherein said detected levels of MMP-8 and CRP are lowered when the amount of MMP-8 is below the predetermined value for MMP-8 and the amount of CRP is below the predetermined value for CRP.

3. (canceled)

4. The method according to claim 1, wherein activated MMP-8 is detected.

5. The method according to claim 1, wherein said cardiovascular disease is selected from the group consisting of cardiovascular disease (CVD), coronary artery disease (CAD), angina pectoris, acute myocardial infarction (AMI), stroke, hypertensive heart disease, rheumatic heart disease, cardiomyopathy, atrial fibrillation, congenital heart disease, endocarditis, aortic aneurysms, and peripheral artery disease.

6-8. (canceled)

9. The method according to claim 1, wherein the sample is serum, plasma or whole blood.

10. The method according to claim 9, wherein the sample is serum.

11. The method according to claim 1, wherein detection of MMP-8 is performed with immunoassay.

12. The method according to claim 1, wherein detection of CRP is performed with immunoassay.

13. The method according to claim 11, wherein said immunoassay is one or more selected from the group consisting of ELISA, IFMA, lateral flow and microfluidics based point-of-care (PoC) assays, turbidimetry, nephelometry, particle enhanced turbidimetry, particle enhanced nephelometry and latex agglutination.

14-15. (canceled)

16. The method according to claim 12, wherein said immunoassay is one or more selected from the group consisting of ELISA, IFMA, lateral flow and microfluidics based point-of-care (PoC) assays, turbidimetry, nephelometry, particle enhanced turbidimetry, particle enhanced nephelometry and latex agglutination.

17. The method according to claim 1, wherein the treatment for cardiovascular disease is selected from lifestyle changes, invasive procedures, and combinations thereof.

18. The method according to claim 17, wherein the invasive procedure is revascularizations cardiac rehabilitation.

19. The method according to claim 1, wherein the medication is selected from the group consisting of antiplatelets, statins, beta-blockers, nitrates, angiotensin-converting enzyme (ACE) inhibitors, angiotensin II receptor antagonists, calcium channel blockers, diuretics, and doxycycline.

20. The method according to claim 19, wherein statins are selected from atorvastatin, simvastatin, rosuvastatin, and pravastatin.

21. The method according to claim 19, wherein the beta-blockers are selected from atenolol, bisoprolol, metoprolol, and nebivolol.

22. The method according to claim 19, wherein the ACE inhibitors are selected from ramipril and Lisinopril.

23. The method according to claim 19, wherein doxycycline medication reduces elevated CRP and MMP-8 and MMP-9 levels in plasma or serum.
